(19) United States
(12) Patent Application Publication
(10) Pub. No.: US 2001/0048951 A1
(43) Pub. Date: Dec. 6, 2001

(54) Methods of administering a synthetic plasma like solution, and systems and kits for use in practicing the same

(76) Inventor: Hal Sternberg, Berkeley, CA (US)

Correspondence Address: Bret E. Field, Bozicevic, Field and Francis LLP, Suite 200, 200 Middlefield Road, Menlo Park, CA, 94025, US

(21) Appl. No.: 09/834,747

(22) Filed: Apr. 13, 2001

Related U.S. Application Data
(60) Provisional application No. 60/197,307, filed on Apr. 14, 2000.

(51) Int. Cl. A61K 33/14; A61K 31/70

(52) U.S. Cl. 424/679; 514/23; 424/680

(57) ABSTRACT

Improved methods of administering a synthetic plasma-like solution to a subject, as well as systems and kits for practicing the same, are provided by the subject invention. In the subject methods, the CO₂ level of the subject, particularly the CO₂ level of at least one of the blood and brain of the subject, is reduced prior to administration of the synthetic plasma-like solution. The subject methods find use in a variety of applications where synthetic plasma-like solutions are employed, including the treatment of hypovolemia, hyphemia, and surgical procedures in which at least a portion of a subject's blood is replaced with a synthetic plasma-like solution and provide for a number of improvements, including a reduced risk of acidosis/acidemia and complications associated therewith.

### CROSS REFERENCED TO RELATED APPLICATIONS

Pursuant to 35 U.S.C. § 119 (e), this application claims priority to the filing date of the U.S. Provisional Patent Application Ser. No. 60/197,307 filed Apr. 14, 2000; the disclosure of which are herein incorporated by reference.

### INTRODUCTION

1. Technical Field

The technical field of this invention is plasma substitute solutions.

2. Background of the Invention

Plasma substitute solutions, or synthetic plasma-like solutions, find use in a variety of different applications in the medical, biomedical research and related fields. For example, physiologically acceptable solutions find use as plasma substitutes in surgical applications that require the replacement of significant amounts of blood plasma volume. Such applications include treatments for blood lost during surgery or trauma, or when a tissue, organ, group of organs or an entire subject needs to be maintained at a hypothermic or frozen state. Such applications also include applications in which a patient s blood is flowed through an external device, such as a cardiopulmonary bypass machine, where the extra circulatory volume space resulting from attachment of the patient s circulatory system to the device must be filled with a compatible blood substitute, i.e. blood volume expander.

Because of their importance in a variety of different applications, as indicated above, a wide variety of different synthetic plasma like solutions have been developed over the years. Various physiologically acceptable solutions, particularly blood substitute solutions, and methods for their use are described in U.S. Pat. Nos. RE 34,077; 3,937,821; 4,001,401; 4,061,736; 4,216,205; 4,663,166; 4,812,310; 4,908,350; 4,923,442; 4,927,806; 5,082,831; 5,084,377; 5,130,230; 5,171,526; 5,210,083; 5,274,001; 5,374,624; and 5,407,428.

While the field of synthetic plasma like solutions is somewhat developed in terms of different types of solutions and methods for their use, there is a continued need for the development of improved methods of using such solutions which lead to better clinical results than are achievable today.

Relevant Literature

Various physiologically acceptable solutions, particularly blood substitute solutions, and methods for their use are described in U.S. Pat. Nos. RE 34,077; 3,937,821; 4,001,401; 4,061,736; 4,216,205; 4,663,166; 4,812,310; 4,908,350; 4,923,442; 4,927,806; 5,082,831; 5,084,377; 5,130,230; 5,171,526; 5,210,083; 5,274,001; 5,374,624; 5,407,428; 6,110,504; 6,080,538; 5,968,726; 5,945,272; 5,747,071; 5,733,894; 5,723,281; 5,702,880; 5,698,536; 5,613,944; 5,574,019; and 5,571,801.

### SUMMARY OF THE INVENTION

Improved methods of administering a synthetic plasma-like solution to a subject, as well as systems and kits for practicing the same, are provided by the subject invention. In the subject methods, the CO₂ level of the subject, particularly the CO₂ level of at least one of the blood and brain of the subject, is reduced prior to and/or during administration of the synthetic plasma-like solution. The subject methods find use in a variety of applications where synthetic plasma-like solutions are employed, including the treatment of hypovolemia, hyphemia, and surgical procedures in which at least a portion of a subject's blood is replaced with a synthetic plasma-like solution.

### DESCRIPTION OF THE SPECIFIC EMBODIMENTS

Improved methods of administering a synthetic plasma-like solution to a subject, as well as systems and kits for practicing the same, are provided by the subject invention. In the subject methods, the CO₂ level of the subject, particularly the CO₂ level of at least one of the blood and brain of the subject, is reduced prior to and/or during administration of the synthetic plasma-like solution. The subject methods find use in a variety of applications where synthetic plasma-like solutions are employed, including the treatment of hypovolemia, hyphemia, and surgical procedures in which at least a portion of a subject's blood is replaced with a synthetic plasma-like solution.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that as used in this specification and the appended claims, the singular forms “a,” “an” and “the” include plural reference unless the context clearly dictates otherwise. Unless defined otherwise all technical and scientific terms used herein have the same meaning as commonly understood to one of ordinary skill in the art to which this invention belongs.

Methods

As indicated above, the subject invention provides improved methods of administering a synthetic plasma-like solution to a subject. The subject methods are improved because they provide an improved outcome in the procedure in which they are employed, as compared to a control in which the subject methods are not practiced. In many embodiments, the improved outcome is manifested in a reduced risk, occurrence, incidence etc., of acidosis/acidemia and complications associated therewith, as described in greater detail below. Specific examples of improvements that are obtainable using the subject methods are reviewed below.

In the subject methods, the CO₂ level of the subject is reduced prior to and/or during administration of the synthetic plasma like solution. Where the CO₂ level is reduced prior to administration of the synthetic plasma like solution, the subject is pre-treated by lowering the CO₂ level of the subject, before treatment with the synthetic plasma like solution. This CO₂ reduction pre-treatment of these embodiments typically occurs less than 60 minutes, usually less than 30 minutes and more usually less than 10 minutes prior to administration of the synthetic plasma like solution, where the time period between reduction in CO₂ level and administration of the synthetic plasma like solution may not exceed 5 minutes, 3 minutes or in some embodiments 1 minute. As indicated above, in yet other embodiments the CO₂ level id reduced during administration of the plasma-like solution.

The CO₂ level of a subject is considered to be reduced for purposes of the subject invention if the amount of CO₂ in at least one tissue, e.g., brain, heart, etc., and/or blood, is reduced by an amount which is sufficient to at least reduce, and often prevent, inschemic pathology during the procedure that the subject is undergoing. Of particular interest in many embodiments is a reduction that is sufficient to reduce the incidence or occurrence of acidosis/acidemia and complications associated therewith. In many embodiments the amount of reduction is at least about 5%, usually by at least about 10% and more usually by at least about 20%, as compared to a control, e.g., an identical or substantially identical subject not treated to reduce CO₂ levels. In many embodiments, the amount of CO₂ present in arterial blood is reduced by at least about 5 mm Hg, usually by at least about 10 mm Hg. Thus, in situations where the host initially presents with above normal CO₂ levels, e.g., 50, 60, etc. or higher mm Hg, the CO₂ level may be reduced to 40 mm Hg or lower. Where the presence of a reduction is manifested by a reduction in the amount of CO₂ present in the arterial blood and the host presents with normal CO₂ levels, the CO₂ level in the blood is typically reduced to a level that is below about 35 mm Hg, usually below about 30 mm Hg, and in certain embodiments below about 25 mm Hg. As such, in many embodiments, the subject methods include a first step of reducing the CO₂ level of the blood to a level that prevents ischemic injury, e.g., to a level that is below about 35 mm Hg, usually below about 30 mm Hg, and in certain embodiments below about 25 mm Hg.

The level of CO₂ in the subject may be reduced using any convenient protocol, where both mechanical and pharmacological means may be employed, where the particular protocol employed may include a combination of both mechanical and pharmacological means. Mechanical means of interest are those means that involve the use of external devices or machinery to reduce the CO₂ level of the subject. As such, mechanical means of interest include mechanical ventilation systems/devices. In other words, of interest are respiratory means of reducing CO₂ levels, such as ventilation with oxygen/in air 20-100% O₂ using appropriate inspiratory volumes and rates (breaths/minute). Such respiratory methods are well known to the those of skill in the art and can be readily employed to reduce the CO₂ level of the subject to the desired level without practicing undue experimentation. Also of interest are bypass devices and external oxygenators, which can be employed to remove CO₂ from the blood and other tissues of the subject. Also of interest are hypothermia producing means, which can be employed to slow metabolism in an amount sufficient to provide for the desired reduction in CO₂ levels. A variety of different hypothermia inducing devices are known and available, including cooling blankets, and the like. In the subject methods, a single mechanical CO₂ level reducing means may be employed, or a combination of two or more distinct mechanical means may be employed.

Also of interest are pharmacological means of reducing the CO₂ level of the subject. Pharmacological means of interest include any means in which a pharmacological agent is employed to achieve the desired reduction in CO₂ level. Pharmacological agents of interest include, but are not limited to: 1) anesthetics, pain killers, and muscle relaxants e.g., pancuronim bromide, which serve to reduce movement, activity and CO₂ generation; 2) agents that increase blood pressure and flow of blood through lungs, e.g., dopamine, epinephrine etc., and the like, which serve to remove CO₂ and thereby reduce CO₂ levels; 3) agents that slow metabolic rate to reduce the generation of CO₂, e.g., in a hypothermic plasma like solution blood substituted subject, the introduction of high potassium and magnesium into the plasma like solution perfusate before circulatory arrest provides for a reduction of build up of CO₂; 4) heparin or anticoagulant or blood dilution agents which serve to improve blood flow helping to reduce ischemia and CO₂ accumulation; and the like. Where pharmacological means are employed to reduce CO₂ levels, a single pharmacological means may be employed or two or more different pharmacological agents may be employed together.

As indicated above, both mechanical and pharmacological means may be employed in concert to achieve the desired amount of CO₂ level reduction in the subject. For example, mechanical ventilation can be used to lower CO₂ along with muscle relaxants. Alternatively, normal to high blood pressure can be maintained prior to circulatory arrest or circulatory reduction and administration of the plasma like solution, described in greater detail below. This can be accomplished with plasma volume expansion and/or pressor agents such as epinephrine, dopamine or phenylephrine etc. The increased perfusion pressure allows for faster removal of CO₂ from the brain and other tissues in addition to allowing for faster removal of CO₂ by the lungs (due to increased blood flow rate). In certain embodiments, the pre-treatments include: (1) the introduction of pancuronium bromide into a subject (to relax muscles, prevent shivering and CO₂ generation) prior to blood substitution or circulatory arrest (2) mechanical ventilation to help maintain below normal CO₂ (20-30 mm Hg) levels and above normal arterial oxygen (300 mmHg and higher) for extended periods of time (greater than 30 min); (3) administration of pressure agents such as phenylephrine to raise blood pressures in anesthetized subjects to that of normal or above (i.e., above 80 mm Hg MAP-mean arterial pressure) for extended periods prior to blood replacement with Hextend, lowering of body temperature, and circulatory arrest.

In the subject methods, following reduction in the CO₂ level of the subject as described above, a synthetic plasma-like solution is introduced to, i.e., administered to, the subject. By synthetic is meant man-made or not naturally occurring. A variety of such solutions are known in the art. Solutions of interest include, but are not limited to the solutions described in U.S. Pat. Nos. RE 34,077; 3,937,821; 4,001,401; 4,061,736; 4,216,205; 4,663,166; 4,812,310; 4,908,350; 4,923,442; 4,927,806; 5,082,831; 5,084,377; 5,130,230; 5,171,526; 5,210,083; 5,274,001; 5,374,624; 5,407,428; 6,110,504; 6,080,538; 5,968,726; 5,945,272; 5,747,071; 5,733,894; 5,723,281; 5,702,880; 5,698,536; 5,613,944; 5,574,019; and 5,571,801; the disclosures of which are herein incorporated by reference.

In certain preferred embodiments of the subject invention, the aqueous solutions employed in the subject methods are physiologically acceptable, by which is meant that the solutions may be introduced into the vasculature of a host without inherently causing a toxic reaction. The solutions will have a pH ranging from about 4 to 10, usually from about 4.5 to 9 and more usually from about 5 to 8.5.

The solutions employed in these embodiments include a plurality of electrolytes, including: sodium ion, chloride ion, potassium ion and calcium ion, and optionally magnesium ion. The sodium ion concentration of the solutions ranges from about 70 to 160, usually from about 110 to 150, and in some embodiments from 130 to 150 mM. The concentration of chloride ion in the solution ranges from about 70 to 170, usually from about 80 to 160, more usually from about 100 to 135 and in some embodiments from about 110 to 125 mM. The concentration of potassium ion ranges from the physiological to subphysiological, where by “physiological” is meant from about 3.5 to 5, usually from about 4 to 5 mM, and by “subphysiological” meant from about 0 to 3.5, usually from about 2 to 3 mM, where in many embodiments of the invention, the amount of potassium ion ranges from about 1 to 5, usually from about 2-3 mM, where in certain embodiments, the amount of potassium ion may be higher than 5 mM and range as high as about 5.5 mM or higher, but usually does not exceed about 5.5. mM. The solutions also include calcium ion in an amount ranging from about 0.5 to 6.0 mM, and in many embodiments will range from about 0.5 to 4.0, usually from about 2.0 to 2.5 mM, but in certain embodiments will range from about 4.0 to 6.0, usually from about 4.5 to 6.0 mM. Optionally, the solutions may further include magnesium. When present, the magnesium ion ranges from about 0 to 10 mM, usually from about 0.3 to 3.0 and more usually from about 0.3 to 0.45 mM.

In certain embodiments, the subject solutions include elevated or high levels of both potassium and magnesium, e.g. the high potassium/magnesium fluid introduced to a perfusate to reduce CO₂ levels, as described above. By elevated levels is meant a potassium ion concentration in an amount ranging from about 50 mM to 3.0 M, usually from about 200 mM to 2.5 M, and more usually from about 1.0 to 2.5 M, and a magnesium ion concentration of from about 40 mM to 1.0 M, usually from about 0.1 to 0.9 M and more usually from about 0.3 to 0.7 M.

Also of interest are solutions that include elevated levels of potassium and magnesium electrolytes (known as “super charger solutions”). By elevated levels is meant a potassium ion concentration in an amount ranging from about 50 mM to 3.0 M, usually from about 200 mM to 2.5 M, and more usually from about 1.0 to 2.5 M, and a magnesium ion concentration of from about 40 mM to 1.0 M, usually from about 0.1 to 0.9 M and more usually from about 0.3 to 0.7 M. Theses solutions may further comprise, in certain embodiments, bicarbonate, where the bicarbonate will be present in amounts ranging from about 0.1 to 40 mM, usually from about 0.5 to 30 mM and more usually from about 1 to 10 mM.

The solutions also include a dynamic buffering system, where the term dynamic buffering system is used to refer to one or more reagents that work in combination to keep the pH of the solution in a certain range in an in vivo environment. Preferably, the reagent members of the dynamic buffering system are normal biological components that maintain in vivo biological pH. The dynamic buffering system concept rests on the discovery by the inventors that compounds with no intrinsic buffering capacity in the biological range, such as lactate, acetate, or gluconate which are capable of being metabolized in vivo, act with other solution components to maintain a biologically appropriate pH in an animal, even at hypothermic temperatures and at essentially bloodless conditions. The dynamic buffering system of the present invention depends in part on oxygenation and removal of carbon dioxide (CO₂). The dynamic buffer of the invention has no or substantially no ability to act as a buffer outside of a biological system, i.e., a dynamic buffer maintains pH in the biological range in vivo but not in a cell free environment.

A feature of the dynamic buffering system of the invention is a carboxylic acid, salt or ester thereof. By a carboxylic acid, salt or ester thereof is meant a compound having the general structural formula RCOOX, where R is an alkyl, alkenyl, or aryl, branched or straight chained, containing 1 to 30 carbons which carbons may be substituted, and preferably one of the carbon chains that compose the carbon chain of lactate, acetate, gluconate, citrate, pyruvate, or other biological metabolites; and X is hydrogen or sodium or other biologically compatible ion substituent which can associate at the oxygen position.

Optionally, the dynamic buffering system may further include a source of bicarbonate, usually sodium bicarbonate (NaHCO₃). When present, the concentration of NaHCO₃ will range from about 0.1 mM to 40 mM, usually from about 0.5 mM to 30 mM, and more usually from about 1 mM to 10 mM.

The solution of the present invention does not include a conventional biological buffer. By “conventional buffer” is meant a compound that in solution, in vitro, maintains pH at a particular range. By “conventional biological buffer” is meant a compound which in a cell-free system maintains pH in the biological range of 7-8. Examples of conventional biological buffers include N-2-Hydroxyethylpiperazine-N′-2-hydroxypropanesulfonic acid (HEPES), 3-(N-Morpholino) propanesulfonic acid (MOPS), 2-([2-Hydroxy-1,1-bis(hydroxymethyl)ethyl]amino)ethanesulfonic acid (TES), 3-[N-tris(Hydroxy-methyl) ethylamino]-2-hydroxyethyl]-1-piperazinepropanesulfonic acid (EPPS), Tris[hydroxymethyl]-aminomethane (THAM), and Tris[hydroxymethyl]methyl aminomethane (TRIS). Conventional biological buffers have a pK in the physiological range and function most efficiently in this range. Therefore, these buffers function independently of normal biological processes and are most potent in cell-free systems.

The absence of a conventional biological buffer in the solution of the invention confers several important medical advantages. For example, lower concentrations of buffers consisting of normal biological components are required to maintain in vivo pH, compared to conventional biological buffers. Conventional biological buffers may also pose toxicity problems. Further, the absence of a biological buffer allows the solution to be terminally heat sterilized. Generally, medical solutions are preferred to be terminally heat sterilized prior to use in a patient. The term “terminally heat sterilized” or “heat sterilized” as used herein refers to the process involving heating a solution to about 120° C. for 15 minutes under pressure, i.e., maintaining heat and pressure conditions for a period of time sufficient to kill all or substantially all bacteria and inactivate all or substantially all viruses in the solution. This procedure is normally performed in an autoclave, and is also known as “autoclaving”. The purpose of heat sterilization is to kill possible infectious agents present in the solution. Infectious agents are known to tolerate temperatures up to 100° C. It is generally considered by the art that heating a solution under pressure to 120° C. for about 15 minutes is sufficient to insure sterility.

The solutions also include an oncotic agent. The oncotic agent is made up of molecules whose size is sufficient to prevent its loss from the circulation by readily traversing the fenestrations of the capillary bed into the interstitial spaces of the tissues of the body. As a group, oncotic agents are exemplified by blood plasma expanders. Compounds finding use as oncotic agents in the subject invention may be natural or synthetic, and will usually be polymeric compositions having an average molecular weight of at least about 40,000, usually at least about 100,000 and more usually at least about 200,000, where oncotic agents having a molecular weight of 300,000 or higher may find use. Examples of oncotic agents suitable for use in the solution of the present invention include proteinaceous compounds, such as albumin, e.g. human serum albumin, and cross-linked or high molecular weight hemoglobin, polysaccharides such as glucan polymers, and the like; organic polymers, e.g. PVP, PEG, etc.; and the like; where non-antigenic polysaccharides are preferred;

Polysaccharides that find use as oncotic agents in the subject solutions include hydroxyethyl starches, hydroxymethyl alpha (1→4) or (1→6) polymers, D-glucose polymers, e.g. dextrans having an alpha (1→6) linkage, cyclodextrins, hydroxypropylstarches, hydroxyacetylstarches, and the like.

Hydroxyethyl starches are of particular interest for certain embodiments of the subject invention. The average molecular weight of hydroxyethyl starches finding use in the subject invention may range from 10,000 d to 1,000,000 d or higher, where the molecular weight will typically range from about 40,000 d to 1,000,000 d, usually from about 100,000 to 900,000, and more usually from about 200,000 to 800,000. Preferred are compositions in which the average molecular weight of the hydroxyethyl starch oncotic agent ranges from about 50,000 d to 1,000,000 d, usually from about 100,000 to 900,000 and more usually from about 200,000 to 800,000.The degree of substitution will range from about 4 to 10, where in certain embodiments, the degree of substitution will range from 7 to 10, in other embodiments will range from 4 to 5, and in other embodiments will range from 6 to 7. Therefore, one class of preferred solutions will comprise a hydroxyethyl starch with between about 6 and 7 hydroxyethyl groups for every 10 glucose units. Another class of preferred solutions will comprise between about 4 and 5 hydroxyethyl groups for every 10 glucose units. Yet another class of preferred solutions will comprise between about 7 and 8 hydroxyethyl groups for every 10 glucose units.

A particularly preferred oncotic agent is Hetastarch (McGaw, Inc.), an artificial colloid derived from a waxy starch composed almost entirely of amylopectin with hydroxyethyl ether groups introduced into the alpha (1→4) linked glucose units and having a molar substitution of about 0.7 hydroxyethyl groups/glucose unit. The colloid properties of a 6% solution (wt/wt) of Hetastarch approximates that of human serum albumin.

Another particularly preferred oncotic agent is Pentastarch, which has a molar substitution of about 0.45 hydroxyethyl groups/glucose unit and an average molecular weight range (as measured by the HPSEC method as reported in PDR 1996) of from about 150,000 to 350,000 d, with 80% between 10,000 and 2,000,000 d.

Another particularly preferred oncotic agent is “Hexastarch,” which has a molar substitution of about 0.64 hydroxyethylgroups/glucose unit and an average molecular weight of about 220,000.

In certain embodiments, the hydroxyethyl starch will be a select fraction of the initial hydroxyethyl starch source, particularly a select size fraction, where generally the fraction will be at least one of the fraction having an average molecular weight of less than about 1,000,000 daltons or the fraction having an average molecular weight of greater than about 50,000 daltons. Conventional fractionation means may be used to prepare such fractions.

The concentration of oncotic agent in the solution is sufficient to achieve (when taken together with chloride salts of sodium, calcium and magnesium, organic ion from the organic salt of sodium and hexose sugar discussed above) colloid osmotic pressure approximating that of normal human serum, about 28 mm Hg. Generally, the amount of oncotic agent in the solution will range from about 0.5 to 30, usually from about 1 to 25 and more usually from about 2 to 8%. Where the oncotic agent is a hydroxyethyl starch, the amount present in the solution will range from about 1 to 30, usually from about 2 to 15 and more usually from about 4 to 8%.

In one aspect of the invention, the solution contains two or more oncotic agents with differential clearance rates. The solutions of the present invention having two or more oncotic agents with differential clearance rates provide additional advantages in restoring blood oncotic pressure in a hypovolemic subject over an extended period of time, while encouraging the subject's own production of plasma proteins. Artificial oncotic agents with relatively slow clearance rates include high molecular weight Hetastarch (molecular weight 300,000-1,000,000) and dextran 70, measured to have intravascular persistence rates of 6 hours (Messmer (1989) Bodensee Symposium on Microcirculation (Hammersen & Messmer, eds.), Karger, N.Y., pg. 59). Artificial oncotic agents with relatively fast clearance rates include low and medium molecular weight Hetastarch (average molecular weight 40,000-200,000) and dextran 40, having intravascular persistence rates of 2-3 hours (Messmer (1989) supra).

The solution may further include one or more different optional agents which may be included in the solution to make the solution suited for a particular application. One optional agent that may be included, and usually is included, is sugar. The sugar will generally be a hexose sugar, such as glucose, fructose and galactose, of which glucose is preferred. In the preferred embodiment of the invention nutritive hexose sugars are used and a mixture of sugars can be used. The sugar is typically, though not necessarily, present in the solution in a physiological amount. By the term “physiological amount” or “physiological levels” is meant the concentration of sugar is in a range between 2 mM and 50 mM with concentration of glucose of 5 mM being preferred. At times, it is desirable to increase the concentration of hexose sugar in order to lower fluid retention in the tissues of a subject. Thus the range of hexose sugar may be expanded up to about 50 mM or even above, but usually not above 60 and more usually not above 55 mM, if necessary to prevent or limit edema in the subject under treatment, except where the agent is present as a cryoprotective agent.

The solutions of the present invention may include a blood clotting factor able to accelerate or promote the formation of a blood clot. Preferred blood clotting factors for use in the solution of the invention include vitamin K, Factors I, II, V, VII, VIII, VIIIC, IX, X, XI, XII, XIII, protein C, von Willebrand factor, Fitzgerald factor, Fletcher factor, and a proteinase inhibitor. The concentration of the blood clotting factor is determined by one skilled in the art depending on the specific circumstances of treatment. For example, generally when vitamin K is administered, its concentration will be sufficient to deliver 5-10 mg to the patient.

The solutions of the present invention may include an oxygen-carrying component in a concentration sufficiently low so as not to be toxic to the subject. The oxygen carrying component will usually be present in a sufficient amount to deliver enhanced oxygen to the tissues of a subject without resulting in toxicity to the subject. A “sufficient amount” of an oxygen-carrying component is an amount allowing a resting subject with an unimpaired circulation and physiology to survive and recover from trauma, illness or injury. In normal humans at normal body temperature, this is at least 5-6 ml O₂/100 ml of intravascular fluid. Oxygen-carrying components include hemoglobin extracted from human and non-human sources, recombinant hemoglobin, hemocyanin, chlorocruorin and hemerythrin, and other naturally occurring respiratory pigments extracted from natural sources or made by recombinant DNA or in vitro methods. These compounds may be modified by a number of means known to the art, including by chemical crosslinking or covalent bonding to polyethylene glycol group(s). When the oxygen-carrying component is hemoglobin, it is preferably present in the concentration range of between about 20-200 g/l.

The solutions may further include one or more cryoprotective agents, where by cryoprotective agent is meant any agent that preserves the structural integrity of tissue under hypothermic, e.g. sub-zero, conditions, where in certain embodiments the cryoprotective agent will be an agent that disrupts, at least to a partial extent, the ordered crystal arrangement of water molecules in a manner such that the freezing point of the aqueous solution comprising the cryoprotective agent is lowered as compared to the freezing point of an analogous solution that does not comprise a cryoprotective agent. Cryoprotective agents of interest include: alcohols, particularly low molecular weight aliphatic alcohols, usually C1 to C6 alcohols, more usually C1 to C4 alcohols, such as methanol, ethanol, and the like; polyols, including linear, branched and cyclic polyols, usually low molecular weight aliphatic polyols, including diols, triols, and other polyols, such as sugars (described in greater detail below) where polyols of particular interest include diols, such as ethylenediol, propanediol, butanediol, triols, e.g. glycerol, and the like; sugars, including erythrose, threose, ribose, arabinose, xylose, lyxose, allose, atrose, glucose, mannose, gulose, idose, galactose, talose, erythrulose, ribulose, xylulose, psicose, fructose, sorbose, tagatose and disaccharides, e.g. sucrose, lactose and maltose, where glucose is particularly preferred; other agents such as timethylamine, trimethylamine oxide (TMAO), DMSO, urea, formamide, dimethylformamide and the like; clathrates, silicon comprising agents, such as silanes and the like, fluorocarbon compounds and derivatives thereof; etc; where the cryoprotective agent may be forced into solution by pressure and/or a suitable surfactant agent may be employed, where such surfactant agents are known to those of skill in the art. Such agents will typically be present in amounts sufficient to provide the desired cryoprotective effect, where the particular amount of the agent will depend on the particular agent employed. When the agent is a polyol, e.g. a diol, it will generally be present in amounts ranging from about 0.2 to 1 M or 0 to 30%. With respect to propanediol, in particular a range of 0.2 M to 0.6 M is preferred and a concentration of about 0.4 M propanediol is most preferred. 1,2 propanediol is preferred as the adduct to the solution used for low temperature organ and donor preservation according to the invention, although 1,3 propanediol may be used. For TMAO, TMAO will be present in the solution in a final concentration in a range between 0.2 M and 7M. When glycerol is employed, it will be present in a concentration ranging from about 0 to 40%, usually from about 5 to 30%, and more usually 5 to 20%. When DMSO is employed, it will be present in amounts ranging from about 0 to 40%, usually from about 5 to 30%, and more usually from about 5 to 20%. When a sugar is employed (particularly glucose), the sugar ranges between about 0.6 M to about 1.4 M, with 1.0 M being preferred for certain embodiments.

In one class of preferred embodiments, the solutions employed in the subject methods are one of the following:

Solution A

| | |
|---|---|
| High Molecular Weight Hetastarch | 1 to 10% w/v |
| (average molecular wt. of 350,000-900,000) | |
| Ca++ | 1-6 mM |
| K+ | 1-5 mM |
| Mg++ | 0-10 mM |
| lactate | 1-40 mM |
| glucose | 0-50 mM |

Solution B

| | |
|---|---|
| High Molecular Weight Hetastarch | 1 to 10% w/v |
| (average molecular wt. of 350,000-900,000) | |
| Ca++ | 1-6 mM |
| K+ | 1-5 mM |
| Mg++ | 0-10 mM |
| lactate | 1-40 mM |
| glucose | 0-50 mM |
| bicarbonate | 5-10 mM |

Cryoprotective Solutions

| | | |
|---|---|---|
| I. | High Molecular Weight Hetastarch | 1 to 10% w/v |
| | (average molecular wt. of 350,000-900,000) | |
| | Ca++ | 1-6 mM |
| | K+ | 1-5 mM |
| | Mg++ | 0-10 mM |
| | lactate | 1-40 mM |
| | glucose | 0-50 mM |
| | bicarbonate | 5-10 mM |
| | glycerol | 10-20% |
| II. | High Molecular Weight Hetastarch | |
| | (average molecular wt. of 350,000-900,000) | 1 to 10% w/v |
| | Ca++ | 1-6 mM |
| | K+ | 1-5 mM |
| | Mg++ | 0-10 mM |
| | lactate | 1-40 mM |
| | bicarbonate | 5-10 mM |
| | glycerol | 10-20% |
| III. | High Molecular Weight Hetastarch | |
| | (average molecular wt. of 350,000-900,000) | 1 to 10% w/v |
| | Ca++ | 1-6 mM |
| | K+ | 1-5 mM |
| | Mg++ | 0-10 mM |
| | lactate | 1-40 mM |
| | glucose | 0-50 mM |
| | bicarbonate | 5-10 mM |
| | glycerol | 5-15% |
| | DMSO | 5-15% |

The amount of fluid solution that is administered to the subject in the subject methods may vary, depending on the particular application in which the subject methods are being employed. In general, the amount of plasma like solution that is administered to the subject following CO₂ level reduction, as described above, it at least about 0.25 l, usually at least about 1.0 l and often at least about 2 l, where the amount that is administered to the subject may be as great as 10 l, 50 l or 100 l or greater, depending on the particular application.

The subject methods are suitable for use with a wide variety of different types of subjects or hosts. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Applications

The subject methods find use in a variety of different applications. The subject methods find particular use in applications where it is desired to replace at least a portion of a host's (or tissue or organ thereof) circulating blood volume with a substitute solution, where such applications include: surgical procedures, including procedures involving a reduction in the temperature of a host from the host's normal body temperature; as a blood substitute; to maintain physiological integrity following death; as a cold preservation agent for tissue or organ; in regional chemoperfusion; and the like. General indications in which the subject methods find use include the treatment of hypovolemia (i.e., reduced plasma volume), hyphemia (i.e. oligemia or reduced blood volume), low blood pressure; etc.

In certain applications, the methods are used in situations where the solution is employed as a circulating solution in conjunction with oxygen or hyperbaric oxygen at normal body temperatures or during procedures when the subject's body temperature is reduced significantly below the subject's normal temperature. For example, during surgical procedures and in cadaver organ donation at low temperatures, the subject's blood may be replaced with the cold circulating solution of the invention, where the solution may be circulated for a time to perfuse and maintain the subject and its organs intact during the procedure. Consistent with the above description, the CO₂ level of the subject is reduced prior to administration of the solution.

In these applications, following CO₂ level reduction, the solution may be administered intravenously or intra arterially to a euthermic subject which is placed in a pressurized atmosphere of increased oxygen concentration up to 100% oxygen or to such a subject undergoing a procedure during which the subject's body temperature is reduced significantly below the subject's normal temperature whether or not hyperbaric oxygen is used. While the solution is being administered to and circulated through the subject, various agents such as cardioplegic agents may be administered either directly into the subject's circulatory system, administered directly to the subject's myocardium, or added to the circulating solution of the present invention. These components are added to achieve desired physiological effects such as maintaining regular cardiac contractile activity, stopping cardiac fibrillation or completely inhibiting contractile activity of the myocardium or heart muscle.

Cardioplegic agents are materials that cause myocardial contraction to cease and include anesthetics such as lidocaine, procaine and novocaine and monovalent cations such as potassium ion in concentrations sufficient to achieve myocardial contractile inhibition. Concentrations of potassium ion sufficient to achieve this effect are generally in excess of 15 mM, and magnesium may also be present in amounts in excess of about 0.5 mM.

During revival of a subject (after a period of subnormal temperature or cryogenic maintenance using the solution according to the invention to maintain the subject) the subject may be reinfused with a mixture of the solution according to the invention along with blood retained from the subject or obtained from blood donors. As the subject is warmed, whole blood is infused until the subject achieves an acceptable hematocrit, generally exceeding hematocrits of about 20%. When an acceptable hematocrit is achieved, perfusion is discontinued and the subject is revived after closure of surgical wounds using conventional procedures.

In general, the solution is administered using an intravenous line (when the subject is at normal temperature) or to a chilled subject using a pumped circulating device such as a centrifugal pump, roller pump, peristaltic pump or other known and available circulatory pump. The circulating device is connected to the subject via cannulae inserted surgically into appropriate veins and arteries. When the solution is administered to a chilled subject, it is generally administered via an arterial cannula and removed from the subject via a venous cannula and discarded, stored or circulated.

The subject methods may be used in a variety of surgical settings and procedures. They may be useful in delicate neurosurgery where clear surgical fields are imperative and reduced central nervous system activity may be desirable and achieved by performing the procedure on a patient whose core temperature and/or cerebral temperature has been substantially reduced.

The methods may be used to maintain a subject (which has lost a significant amount of blood, e.g. 20% to 98% of its blood) at normal body temperatures in a pressurized environment at increased oxygen concentration above atmospheric oxygen tension up to 100% oxygen. The subject is maintained in a high oxygen concentration, either continuously or periodically, until enough blood components can be synthesized by the subject to support life at atmospheric pressure and oxygen concentration. The methods may be used to maintain a subject at temperatures lower than normal body temperature and at a reduced rate of metabolism after traumatic life threatening injury until appropriate supportive or corrective surgical procedures can be performed. In addition the methods may be used to maintain a patient having a rare blood or tissue type until an appropriate matching donor can be found and replacement blood units or other organ can be obtained.

Surprisingly it has been discovered that it is possible to replace substantially all of a mammalian subject's circulating blood with the methods and to maintain the subject alive without reinfusing blood into the subject. Substantially all of a mammalian subject's circulating blood is considered to be replaced when the subject's hematocrit drops below 10%. Hematocrit may be lower than 10% if O₂ is provided to the subject, or substantially lower than 10% in a hyperbaric O₂ chamber. The subject methods can of course be used to maintain a subject having a hematocrit in excess of 10%.

The procedure for replacing substantially all of a mammalian subject's circulating blood may be carried out with the mammalian subject's body temperature being maintained at its substantially normal temperature. In addition the procedure may be carried out with cooling of the subject and reduction of the mammalian subject's body temperature below that of its normal temperature. Cooling may be accomplished by chilling the subject in an ice bath, ice-salt slurry, or cooling blanket. The subject may be further cooled by chilling the solution according to the invention prior to perfusing the subject with the solution.

In the procedure according to the invention for replacing substantially all of a mammalian subject's circulating blood, it is preferred that the subject is chilled and perfused with the solution, using an arterial catheter to deliver the solution to the subject's circulatory system and a venous catheter to remove blood and the perfusate from the subject. Substantially all of the subject's circulating blood is removed in this manner as determined by measurement of the hematocrit of the effluent from the venous catheter. When substantially all of the subject's circulating blood is removed, perfusion may be stopped.

In addition, the procedure for replacing substantially all of the subject's blood may be carried out with the aid of hyperbaric O₂. The subject is placed in a hyperbaric chamber pressurized with oxygen at concentrations exceeding 20%, preferably 100% oxygen. The pressure of the hyperbaric chamber is maintained during most of the procedure in a range between 0.5 pounds per square inch over atmospheric pressure to pressures up to about twice atmospheric pressure. In one embodiment, the procedure is performed with the subject in a hyperbaric chamber at hyperbaric pressures of about 0.07 to about 2 atmospheres over ambient pressure (0.5-30 pounds per square inch [psi]) with 100% oxygen. If necessary, the pressure of the hyperbaric chamber may be reduced to atmospheric pressure during wound closure. The subject is subsequently maintained at hyperbaric pressure at high oxygen concentration. The pressure is gradually reduced to a lower pressure but one still hyperbaric. Preferably the pressure is maintained below 10 psi to about 5 psi for a number of hours to several days. Subsequently, the pressure is again gradually lowered below 1 psi and preferably to about 0.5 psi and is maintained at this pressure for an additional period of time up to a day or more.

The methods may also be used to maintain the physiological integrity of an organ donor subject immediately after the occurrence of brain death. The subject can be chilled, the subject's blood removed and replaced with a circulating solution maintained below 37° C., or while circulating cold solution according to the invention. Through this use of the solution, ischemia of vital organs can be minimized. By circulating cold solution according to the invention through the subject's circulatory system at low temperature with or without placing the subject in a hyperbaric oxygen chamber, vital organs can be maintained for longer periods of time, thus maximizing the number of organs that can be effectively used from one donor for potential transplant recipients.

In another aspect of the invention, it has been discovered that by using certain adducts, particularly propanediol and high concentration glucose to augment the solution, it may be possible to reduce the temperature of donor organs, and in particular donor hearts, below the freezing point of water (0° C.) and recover them from freezing in a useful state, i.e. a state capable of maintaining coordinated cardiac contraction. Furthermore by using the solution according to the invention with such adducts, it has been possible to reduce the temperature of intact mammalian subjects below the freezing point of water (0° C.) and restore them from freezing in a state capable of maintaining coordinated cardiac contraction and even respiration and conscious reaction. Other organ systems are also believed to be maintained with a high degree of biological integrity, i.e. in a physiological state capable of maintaining life.

In all of the above representative applications in which a plasma like solution is employed, administering the plasma like solution according to the subject methods results in an improvement in the outcome of the method. The improvement may manifest itself in one or more different ways, including speedier and fuller recovery from the procedure, reduction in lasting side effects, reduced pathology, reduced tissue damage, etc. An example of an improvement that is achieved with the subject methods is described in greater detail in the experimental section infra. One improvement of particular interest is the reduction in the risk of, incidence of or occurrence of acidosis/acidemia following administration of the plasma like solution. The magnitude of the reduction typically is at least about 5%, usually at least about 10% and more usually at least about 20%, where the reduction in risk is determined with respect to a control situation in which a subject is administered a plasma-like solution without prior and/or concomitant CO₂ level reduction.

Kits

Also provided are kits for use in practicing the subject methods, i.e., kits for administering a synthetic plasma-like solution to a subject in need thereof. The subject kits generally include a quantity of a synthetic plasma-like solution. In addition, the subject kits may include a means for reducing the CO₂ level of a subject, e.g., a mechanical means and/or a pharmacological means, as described above. In many embodiments, the kits will further include instructions for use in practicing the subject methods, where the instructions may be present on one more components of the kit, e.g., packaging, package insert, containers holding the solution, etc.

Systems

Also provided are systems for use in practicing the subject methods, i.e., systems for administering a synthetic plasma-like solution to a subject in need thereof. The subject systems generally include a quantity of a synthetic plasma-like solution and a means for reducing the CO₂ level of a subject, e.g., a mechanical means and/or a pharmacological means, as described above.

The following examples are offered by way of illustration and not by way of limitation.

### EXPERIMENTAL

The following example is put forth so as to provide those of ordinary skill in the art with a complete disclosure and description of how to carry out the synthesis of the invention and is not intended to limit the scope of what the inventors regard as their invention. Efforts have been made to ensure accuracy with respect to numbers used (e.g., amounts, temperature, etc.), but some experimental error and deviation should be accounted for. Unless indicated otherwise, parts are parts by weight, molecular weight is weight average molecular weight, temperature is in degrees Centigrade, and pressure is at or near atmospheric.

### EXAMPLE 1

### Solution Compositions.

A. Composition of L solution.

The composition of L solution is as follows: Na⁺ 143 mM; Ca⁺⁺ 2.5 mM; Mg⁺⁺ 0.45 mM; K⁺ mM 3.0; Cl⁻ 124 mM; glucose 5 mM; and lactate 28 mM. The solution is filtered to remove undissolved material and placed in autoclavable containers and heated in an autoclave to a temperature of 120 C for 15 minutes.

B. Composition of HL (BioTime Hextend—lactate) Solution.

L formulation with the addition of 60 g/l of high molecular weight Hetastarch.

C. Composition of HLB (BioTime Hextend—lactate-bicarbonate)

Solution. HL solution with the addition of 5 ml/l 1 M solution of NaHCO₃.

### EXAMPLE 2

### Animal Procedures

A. Test

Revival of a small Hamster using strong ventilation to lower CO₂ level when using the plasma volume expander Hextend to completely replace blood and lower body temperature to near zero.

Protocol: Extended Circulatory and Cardiac Arrest

The following general protocol was extrapolated from experiments performed on 8 animals that were maintained in respiratory, circulatory and cardiac arrest for 4 to 6 hours.

Young female, Golden Syrian hamsters (72-86 g) were anesthetized i.m. with 0.04 mL ketamine (100 mg/mL) and chilled to rectal temperatures of 9-12° C. The animals were then respirated with pure oxygen at pressures ranging between 3 and 4.5 inches of H₂O and which was delivered for 0.1 second at 0.1 second intervals. The brachial artery was cannulated and approximately 0.2 mL of a dilute heparin solution (1 part heparin to 3 parts HLB) as well as 0.35 mL Pancuronium Bromide (0.02 mg/mL) were administered intra-arterially (i.a.). The arterial pH and partial pressures of O₂ and CO₂ were measured and the femoral vein was then cannulated. Animals were maintained at rectal temperatures of approximately 10.5-13° C. during the perfusion of the 4 mL of HLB. Animals were then chilled during the perfusion of an additional 6 mL of cold HLB perfused at a rate of 0.33-0.66 mL/min. After a total perfusion of approximately 10 mL and upon reaching rectal temperatures between 2.5 and 4° C., 0.3-0.75 mL of a 100 mM KCl/30 mM MgSO₄ in Hespan was injected (i.a.). In each case the minimal amount necessary to fully arrest the heart was given. Both the venous and arterial cannulas were then plugged and respiration was discontinued. Animals were maintained in cardiac, circulatory and respiratory arrest for 4-6 hours at temperatures ranging between 0.7-2.0° C. Following this period of stand-still animals were again respirated with pure oxygen at slightly elevated pressures (4-8 inches of H₂O) while simultaneously being and perfused with HLB at a rate of 0.33 mL/min. 10-11 mL were perfused at temperatures of approximately 4-6° C. before beginning the reperfusion with donor blood (12-15 mL). During the reperfusion of blood (0.33-0.66 mL/min) the animals were slowly rewarmed through the use of a heating lamp and a gentle warming of the stage with warm water. Following the spontaneous reappearance of a heartbeat the perfusion rate was accelerated to increase vascular pressure. This pressure is gauged by the swelling or lack there of visible in the jugular vein. Rewarming of the animals was further continued until breathing resumed and animals were revived to consciousness.

Several factors distinguish this achievement which permits the consistent revival of hamsters (7 of 8), from previous failed attempts. These factors include (1) the introduction of Pancuronium Bromide, a neuro-muscular paralytic, which effectively inhibits the generation of CO₂ (2) increased respiratory pressure, which more efficiently removes CO₂. (3) the administration of an anticoagulant facilitated increased perfusion rates and pressures which are also closely linked to the efficient CO₂.

It is evident from the above results and discussion that improved results are obtained when the subject methods are employed to administered plasma like solutions. Significantly, administration of a plasma like solution to a subject according to the subject methods greatly reduces the risk of acidosis/acidemia and complications associated therewith. As such, the subject invention is a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of administering a synthetic plasma-like solution to a subject in need thereof, said method comprising:
(a) reducing the level of CO₂ in said subject; and
(b) administering said plasma-like solution to said subject;
wherein said method results in a reduced risk of acidosis/acidemia.

2. The method according to claim 1, wherein said method comprises reducing at least one of the blood level and brain level CO₂ of said subject.

3. The method according to claim 1, wherein said subject has at least a sub-physiologic blood flow.

4. The method according to claim 1, wherein said subject is in circulatory arrest.

5. The method according to claim 1, wherein said subject suffers from hypovolemia.

6. The method according to claim 1, wherein said subject suffers from hyphemia.

7. The method according to claim 1, wherein said subject suffers from low blood pressure.

8. The method according to claim 1, wherein said subject is undergoing surgery.

9. The method according to claim 8, wherein said surgery is low temperature surgery.

10. The method according to claim 8, wherein said surgery is stopped heart surgery.

11. The method according to claim 8, where said surgery includes replacing at least a portion of the blood of said subject with said synthetic plasma like solution.

12. The method according to claim 1, wherein said CO₂ level is reduced using a mechanical means.

13. The method according to claim 1, wherein said CO₂ level is reduced using a pharmacological means.

14. The method according to claim 1, wherein said synthetic plasma like solution comprises:
electrolytes;
a dynamic buffering system; and
at least one oncotic agent;
wherein said solution does not comprise a biological buffer.

15. The method according to claim 14, wherein said dynamic buffering system comprises bicarbonate.

16. The method according to claim 14, wherein said electrolytes of said plasma-like solution comprise sodium, potassium, calcium, chloride ion and magnesium.

17. The method according to claim 14, wherein said solution further comprises a simple sugar.

18. The method according to claim 1, wherein said synthetic plasma like solution comprises:
sodium, potassium, calcium, chloride ion and magnesium electrolytes;
bicarbonate; and
at least one starch oncotic agent; and
a simple sugar;
wherein said solution does not comprise a biological buffer.

19. A system for administering a synthetic plasma-like solution to a subject in need thereof, said system comprising:
(a) a synthetic plasma-like solution; and
(b) a means for reducing the CO₂ level of a subject.

20. The system according to claim 19, wherein said means is a mechanical means.

21. The system according to claim 19, wherein said means is a pharmacological means.

22. A kit for administering a synthetic plasma-like solution to a subject in need thereof, said system comprising:
(a) a synthetic plasma-like solution; and
(b) a means for reducing the CO₂ level of a subject.

23. The kit according to claim 22, wherein said means is a mechanical means.

24. The kit according to claim 22, wherein said means is a pharmacological means.

25. The kit according to claim 22, wherein said kit further comprises instructions for practicing the method of claim 1.
